(19) United States
(12) Patent Application Publication
(10) Pub. No.: US 2026/0301974 A1
(43) Pub. Date: Oct. 1, 2026

(54) ARTIFICIAL INTELLIGENCE SYSTEMS AND METHODS FOR ENGAGING MEMBERS IN HEALTH BENEFITS

(71) Applicant: RiskAverse Inc., Westborough, MA (US)

(72) Inventors: Kaushik Raj VISWANATHAN, Westborough, MA (US); Zackary Insoo SADLIER, Westborough, MA (US); Michael Edward LLOYD, Westborough, MA (US)

(73) Assignee: RiskAverse Inc., Westborough, MA (US)

(21) Appl. No.: 19/094,376

(22) Filed: Mar. 28, 2025

(51) Int. Cl. G16H 80/00; G16H 10/60

(52) U.S. Cl. CPC G16H 80/00; G16H 10/60

(57) ABSTRACT

Disclosed herein are methods, systems, and computer-readable storage media for targeted outreach messaging new members regarding healthcare information, including receiving new healthcare data of new member(s) at artificial intelligence (AI) model(s) trained based on historical healthcare data and engagement result data of a plurality of historical members; and, for each outreach message: determining, by the AI model(s), a combination of optimal outreach parameters for targeting and engaging the new member based on the new healthcare data of the new member, the historical healthcare data of the plurality of historical members, and the engagement result data of the plurality of historical members; generating an outreach message for engaging the new member based on at least one received message constraint and the combination of optimal outreach parameters; and deploying the outreach message to the new member based on the combination of optimal outreach parameters that targets the new member regarding healthcare information.

### FIELD

This application relates generally to artificial intelligence (AI) systems and methods for engaging members, and, more specifically, to AI systems and methods trained on healthcare data for engaging members in health benefits and services.

### BACKGROUND

Traditional marketing and member communications use a “flood the zone” strategy for engaging members. Since members often ignore communications, vendors often send the same communication to customers and members in several formats hoping that the member will engage with one of the messages. For example, omni-channel marketing solutions use batch email, SMS, phone calls, and postal mail in an attempt to engage the members/customers in the message content. These omni-channel communication methods require communication teams to manually build and manage complex engagement workflows or journeys. Nonetheless, customer engagement with these mass communication methods is less than ideal and can frustrate members that grow tired of repeated communications.

In the healthcare industry in particular, valuable services and other healthcare offerings are available to members, yet healthcare entities fall short in efficiently and effectively engaging new and existing members in these services. For example, communications that require member engagement can include simple requests that can be completed quickly, such as scheduling a flu shot, signing up for an online profile on a portal, to more involved requests that require long term engagement, such as signing up and following a diabetes or dialysis program. Existing solutions for engaging members in these valuable services result in significant customer acquisition cost (CAC) and operational costs for the vendor with minimal success in converting members. Moreover, existing systems severely limit the amount of personalization that can be achieved in engaging members. Healthcare in particular is an industry in need of individualized communication methods tailored to an individual's unique health needs and other personal attributes to actively engage the individual and improve their overall wellbeing.

### SUMMARY

Described are systems and methods for improving online engagement with new customers and members, for example healthcare-related customers and members. The described systems use artificial intelligence (AI) to determine the best manner (e.g., time, tone, cadence, etc.) to communicate with each new member. By determining the best manner to communicate with each member, vendors may improve the marketing efficiency of their communications by communicating with members in a manner that the user is most likely to view and engage with. This can enable vendor to send fewer messages and improve the engagement with the messages that they send. The described systems and methods also improve the member experience since members are not bombarded with messages that they may otherwise ignore.

In the healthcare industry, the AI systems and methods described herein can be used to engage members and customers in health benefits and services. The AI systems described herein are uniquely trained based on historical healthcare data (e.g., demographic data, medical data, etc.) and historical engagement result data. In use, the AI model ingests healthcare data (e.g., demographic data, medical data, etc.) of a new member, and automatically determines the optimal parameters (e.g., time, tone, channel, and/or cadence) for engaging the new member based on the historical healthcare data and engagement result data. The optimal parameters can be refined based on one or more constraints provided by a user of the system to generate a campaign, including an outreach message, for engaging the new member. The system then deploys the campaign (e.g., the outreach message) to engage the new member in the health benefits and services in a way most probable to result in engagement of the new member with the outreach message. Accordingly, the AI systems and methods described herein provide a scalable, efficient solution for automatically creating a unique journey for engaging a particular member based only on the member's healthcare data, the journey for said particular member being different from the next new member. Using the methods and systems described herein, customer acquisition cost (CAC) is lowered, and member conversions are increased for downstream services, leading to larger revenue streams for the business.

In some aspects, an automated method for targeted outreach messaging new members regarding healthcare information is provided, the method comprising: receiving new healthcare data of one or more new members at one or more artificial intelligence (AI) models trained based on historical healthcare data and engagement result data of a plurality of historical members; and for each outreach message: determining, by at least the one or more AI models, a combination of optimal outreach parameters for targeting and engaging the new member based on the new healthcare data of the new member, the historical healthcare data of the plurality of historical members, and the engagement result data of the plurality of historical members; generating an outreach message for engaging the new member based on at least one received message constraint and the combination of optimal outreach parameters; and deploying the outreach message to the new member based on the combination of optimal outreach parameters that targets the new member regarding healthcare information.

In some aspects, a system for targeted outreach messaging new members regarding healthcare information is provided, the system comprising one or more processors; memory; and one or more programs stored on the memory that when executed by the one or more processors cause the one or more processors to: receive new healthcare data of one or more new members at one or more artificial intelligence (AI) models trained based on historical healthcare data and engagement result data of a plurality of historical members; and for each outreach message: determine, by at least the one or more AI models, a combination of optimal outreach parameters for targeting and engaging the new member based on the new healthcare data of the new member, the historical healthcare data of the plurality of historical members, and the engagement result data of the plurality of historical members; generate an outreach message for engaging the new member based on at least one received message constraint and the combination of optimal outreach parameters; and deploy the outreach message to the new member based on the combination of optimal outreach parameters that targets the new member regarding healthcare information.

In some aspects, a non-transitory computer-readable storage medium storing one or more programs for targeted outreach messaging new members regarding healthcare information is provided, the programs for execution by one or more processors of an electronic device that when executed by the device, cause the device to: receive new healthcare data of one or more new members at one or more artificial intelligence (AI) models trained based on historical healthcare data and engagement result data of a plurality of historical members; and for each outreach message: determine, by at least the one or more AI models, a combination of optimal outreach parameters for targeting and engaging the new member based on the new healthcare data of the new member, the historical healthcare data of the plurality of historical members, and the engagement result data of the plurality of historical members; generate an outreach message for engaging the new member based on at least one received message constraint and the combination of optimal outreach parameters; and deploy the outreach message to the new member based on the combination of optimal outreach parameters that targets the new member regarding healthcare information.

In some aspects, the new healthcare data and/or the historical healthcare data comprises clinical data including at least one medical condition, a medication history, at least one laboratory test result, at least one imaging report, and/or an immunization record. In some aspects, the new healthcare data and/or the historical healthcare data comprises at least one of demographic data, geographic data, insurance claim data, behavioral data, psychographic data, and social determinants of health (SDoH) data. In some aspects, the healthcare information comprises information regarding at least one of a medical benefit, a health insurance benefit, a vision benefit, a hearing benefit, a financial benefit, an educational benefit, and a life insurance benefit. In some aspects, the one or more AI models are further trained based at least on historical demographic data of the plurality of historical members. In some aspects, the one or more new members are different from the plurality of historical members.

In some aspects, the combination of optimal outreach parameters comprises a tone of the outreach message, a time at which to send the outreach message, a channel through which to send the outreach message, and/or a cadence at which to send the outreach message. In some aspects, the engagement result data indicates a time at which the plurality of historical members engaged with the outreach message and a time at which the outreach message was sent. In some aspects, the at least one received message constraint comprises a content of the outreach message. In some aspects, the at least one received message constraint comprises a constraint on a time at which to send the outreach message, a constraint on a channel through which to send the outreach message, a constraint on a tone of the outreach message, and/or a constraint on a cadence at which to send the outreach message.

In some aspects, determining the combination of optimal outreach parameters for targeting and engaging the new member comprises using a random forest classification AI model configured for determining a plurality of combinations of outreach parameters for engaging the new member. In some aspects, determining the combination of optimal outreach parameters for targeting and engaging the new member comprises, by a machine learning application programming interface (API) and/or the one or more AI models: assigning a plurality of confidence scores to the plurality of combinations of outreach parameters; and comparing the plurality of confidence scores to determine the combination of optimal outreach parameters.

In some aspects, the automated method comprises: receiving an engagement result from the new member; and storing the engagement result in the engagement result data. In some aspects, the automated method comprises re-training the one or more AI models based on the engagement result of the new member. In some aspects, the automated method comprises, based on a determination that the new member engaged with the outreach message, deploying at least one additional outreach message to the new member based on the combination of optimal outreach parameters. In some aspects, the automated method comprises, based on a determination that the new member did not engage with the outreach message: determining, by the one or more AI models, a new combination of optimal outreach parameters for targeting and engaging the new member based on the new healthcare data of the new member, the historical healthcare data, and the engagement result data, wherein the new combination of optimal outreach parameters is different from the initial combination of optimal outreach parameters; generating a new outreach message for engaging the new member based on the at least one received message constraint and the new combination of optimal outreach parameters; and deploying the new outreach message to the new member based on the new combination of optimal outreach parameters that targets the new member regarding the healthcare information.

In some aspects, the automated method comprises receiving new healthcare data of a first member and a second member of the one or more new members at the one or more AI models; determining, by the one or more AI models, a first combination of optimal outreach parameters for targeting and engaging the first member based on the new healthcare data of the first member, the historical healthcare data, and the engagement result data; generating a first outreach message for engaging the first member based on the at least one received message constraint and the first combination of optimal outreach parameters; deploying the first outreach message to the first member based on the combination of optimal outreach parameters that targets the first member regarding healthcare information; determining, by the one or more AI models, a second combination of optimal outreach parameters for targeting and engaging the second member based on the new healthcare data of the second member, the historical healthcare data, and the engagement result data, the second combination of optimal outreach parameters different from the first combination of optimal outreach parameters; generating a second outreach message for engaging the second member based on the at least one received message constraint and the second combination of optimal outreach parameters, the second outreach message different from the first outreach message; and deploying the second outreach message to the second member based on the combination of optimal outreach parameters that targets the second member regarding healthcare information.

In some aspects, receiving the new healthcare data for the one or more new members at the one or more AI models comprises: receiving the new healthcare data at an engagement application user interface (UI); passing the new healthcare data from the engagement application UI to a machine learning application programming interface (API); and passing the new healthcare data from the machine learning API to the one or more AI models. In some aspects, the automated method comprises, following passing the new healthcare data from the machine learning API to the one or more AI models: passing a plurality of combinations of optimal output parameters from the one or more AI models to the machine learning API; and passing the combination of optimal output parameters determined from the plurality of combinations of optimal output parameters from the machine learning API to the engagement application UI that generates the outreach message for engaging the new member and deploys the outreach message to the new member.

It will be appreciated that any of the variations, aspects, features and options described in view of the systems apply equally to the methods and vice versa. It will also be clear that any one or more of the above variations, aspects, features and options can be combined.

### BRIEF DESCRIPTION OF THE FIGURES

The invention will now be described, by way of example only, with reference to the accompanying drawings, in which:

FIG. 1 shows an exemplary structure diagram of an artificial intelligence (AI)-backed system for engaging members, in accordance with some aspects.

FIG. 2 shows an exemplary training process for the AI model of FIG. 1, in accordance with some aspects.

FIGS. 3A-3I show exemplary graphical user interfaces (GUIs) of a platform for interacting with the AI-backed system of FIG. 1 for engaging members; FIG. 3A shows an exemplary GUI for uploading new member data to the AI-backed system; FIG. 3B shows an exemplary GUI for setting message constraints; FIG. 3C shows an exemplary GUI for generating a campaign for engaging the cohort of members; FIG. 3D shows an exemplary GUI with the first touchpoint of the campaign constrained based on user inputs; FIG. 3E shows an exemplary GUI for adding events, such as a time constraint, to the campaign for engaging the cohort of members; FIG. 3F shows an exemplary GUI with a time constraint added to the campaign for engaging the cohort of members; FIG. 3G shows an exemplary GUI of the list of the cohort of members after generating the campaign and prior to executing the AI model; FIG. 3H shows an exemplary GUI of a summary of the campaign for engaging the cohort of members; and FIG. 3I shows output of the AI model including a list of optimal parameters for uniquely engaging each of the members in the cohort of members, and deliverability status of the touchpoints, in accordance with some aspects.

FIGS. 4A-4B show an automated method for targeted outreach messaging new members regarding healthcare information using the system of FIG. 1; FIG. 4A shows an initial automated method for determining a combination of optimal outreach parameters for targeting and engaging a new member; and FIG. 4B shows a subsequent method for retraining the AI model based on engagement result data of the new member, in accordance with some aspects.

FIG. 5 shows a computer for targeting and engaging a new member, in accordance with some aspects.

### DETAILED DESCRIPTION

Described herein are artificial intelligence (AI) systems and methods that improve upon existing solutions for engaging and targeting members and customers regarding information. The disclosed methods and systems create an efficient, cost-effective way to improve outreach efforts, minimizing the number of touchpoints necessary to engage members in information by determining the best manner through which to uniquely engage each member. Moreover, the provided AI systems and methods remove the requirement for vendors to manually create journeys for hundreds of thousands of members, or, worse, to engage several members by mass communication methods rather than creating unique journeys. Accordingly, by determining the exact manner (e.g., time, tone, content, and/or cadence) that is best for uniquely engaging a particular member, the AI systems and methods described herein can result in significantly increased clickthrough rates and initial activity rates (i.e., open rates) in the enclosed content, and, in turn, increased enrollment in and use of the related services and benefits. For example, the AI systems and methods can increase enrollment in services such as clinical programs, health screenings, financial card incentives, general monthly awareness emails, financial card activation, etc. Moreover, the AI systems and methods described herein minimize customer acquisition costs and operational overhead for the vendor while maximizing revenue streams.

Described herein are methods, systems, and computer-readable storage media that utilize artificial intelligence (AI) model(s) to target and engage members regarding information, such as healthcare, financial, and/or e-commerce information. For example, the systems and methods provided herein can be used to engage members in health services and benefits. The AI model can be trained based on historical healthcare data and engagement result data. The AI model can determine the optimal outreach parameters for uniquely engaging each member based on the historical healthcare data, the engagement result data, and new healthcare data for the new member(s). The optimal outreach parameter including a time at which to send an outreach message, a tone of the outreach message, content of the outreach message, and/or a cadence at which to send the outreach message.

In some embodiments, the AI methods and systems described herein can personalize engagement of members based on a given member's health condition(s) or other attributes. For example, the AI system may learn the best way for engaging with diabetic patients is to send an email message at a particular time, e.g. in the morning, with a particular tone e.g. relating to the patient's struggles. However, patients of a certain age, with other ailments, with other socioeconomic variables, etc. may have different time and tone preferences. For example, the AI system may determine the best way for engaging middle-aged male patients having anxiety is via a text message, at different particular time, e.g., in the afternoon, and at a particular cadence, e.g., weekly. The methods and systems herein are able to consider all of the different features of members and determine the best time, tone, cadence, channel, etc. with which to seamlessly and uniquely engage the member.

Reference will now be made in detail to implementations and embodiments of various aspects and variations of systems and methods described herein. Although several exemplary variations of the systems and methods are described herein, other variations of the systems and methods may include aspects of the systems and methods described herein combined in any suitable manner having combinations of all or some of the aspects described.

In the following description of the various examples, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

The present disclosure in some embodiments relates to a device for performing the operations herein. This device may be specially constructed for the required purposes, or it may comprise a general-purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each connected to a computer system bus. Furthermore, the computing systems referred to in the specification may include a single processor or may be architectures employing multiple processor designs, such as for performing different functions or for increased computing capability. Suitable processors include central processing units (CPUs), graphical processing units (GPUs), field programmable gate arrays (FPGAs), and ASICs.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present disclosure as described herein.

As described herein, the term “artificial intelligence (AI) model” is defined as a computational system designed to process data, recognize patterns, and make decisions or predictions based on learned information. An AI model used herein may also be referred to as a machine learning model, neural network, deep learning model, intelligent algorithm, or other variants thereof.

As used herein, the term “member” (e.g., “new member,” “historical member”) is understood to encompass a member of a list of individuals. The member can be a customer of a vendor, and these terms are used interchangeably to refer to the person being engaged by the AI systems and methods described herein. A “new member” is a member the vendor desires to engage using the AI systems and methods. A “historical member” is a member the vendor has previously engaged using the AI systems and methods.

As used herein, the term “vendor” is understood to encompass any organization, business, or other entity that is offering (e.g., selling, or providing) a service, product, or other content to a member. The vendor can be the user of the systems described herein; therefore, the terms “user” and “vendor” are used interchangeably herein.

Although described herein predominantly with respect to health benefits and services, it is to be understood that the AI systems and methods described herein may be usable in other industries and is not intended to be limited to healthcare applications. For example, AI systems and methods may be usable for financial, e-commerce, school, retirement industries, etc., to name a few. Inputs to the AI model(s) may change based on industry. For example, financial information may be available to generate learnings from in the financial, e-commerce, and/or retirement industries. In most industries, at a minimum, access to basic demographic information is available for the AI systems and methods described herein to operate on and generate predictions from.

The following description details an AI-backed system for targeting and engaging members, followed by a training process for the AI model of the system. Then, exemplary graphical user interfaces (GUIs) for interacting with the AI-backed system are described before detailing the automated method for targeted outreach messaging new members regarding healthcare information. Finally, a computer for executing the systems and methods provided herein is described.

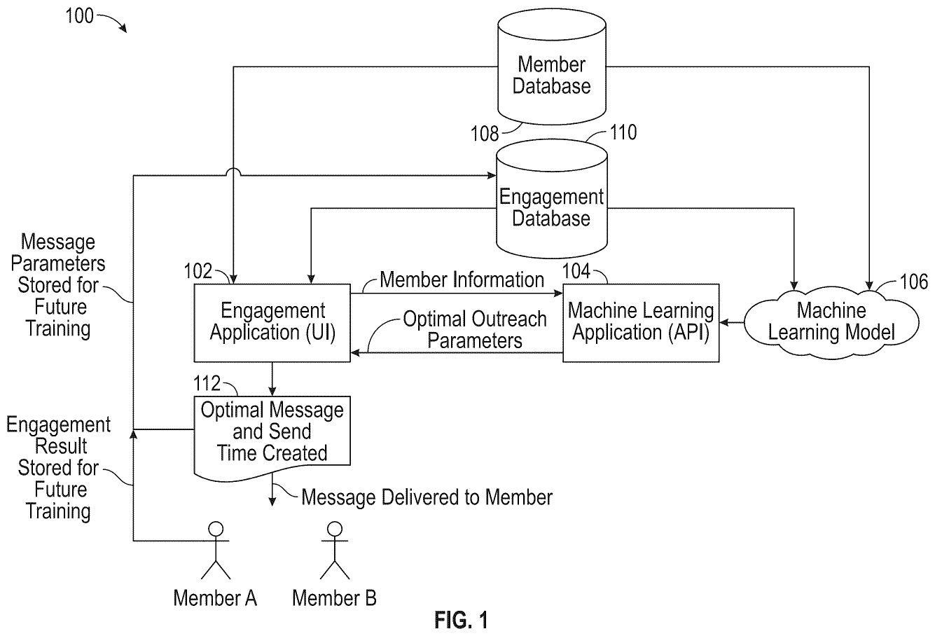

FIG. 1 depicts an AI-backed system 100 for engaging members. In particular, FIG. 1 depicts the flow of data across the system 100, in accordance with some embodiments. The system 100 can include an engagement application user interface (UI) 102, an application programming interface (API, or machine learning application) 104, and one or more AI models 106 (e.g., machine learning models). The system 100 can include a member database 108 and an engagement database 110 that store data for training the AI model 106. Each of these components is described in greater detail below.

The system 100 may include one or more processors, memory, and one or more programs stored on the memory for execution by the one or more processors. For example, the one or more programs can include the engagement application UI 102, the API 104, and the AI model 106 for determining optimal outreach parameters for engaging members.

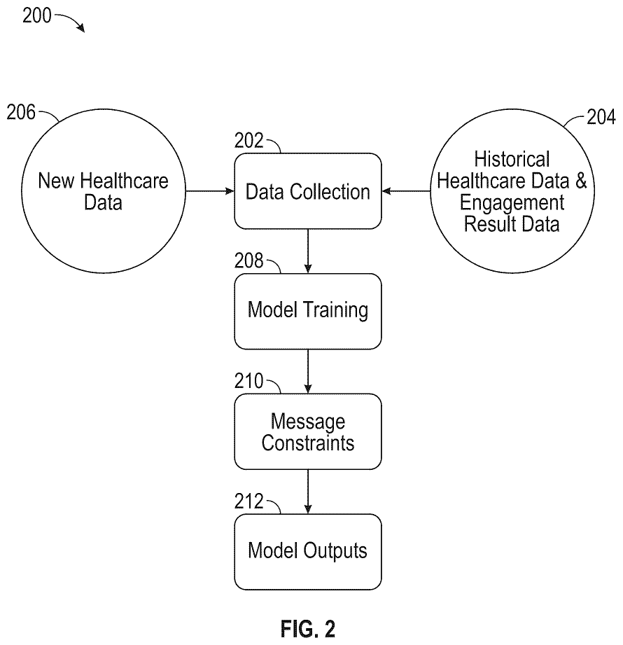

The system 100 can receive new healthcare data for one or more new members at the engagement application UI 102. The new healthcare data can include clinical data, demographic data, geographic data, insurance claim data, behavioral data, psychographic data, and/or social determinants of health (SDoH) data for each new member of a cohort of new members. In some examples, the new healthcare data are demographic data. In some examples, the new healthcare data are demographic data and/or clinical data. Demographic data can include age, gender, race or ethnicity, marital status, and/or education level of the new member. Clinical data can include medical condition(s), a medication history, at least one laboratory test result, at least one imaging report, and/or an immunization record of the new member. Additional examples the various types of data that can be included in the new healthcare data are described below in greater detail with respect to FIG. 2. The system 100 can also receive contact information (e.g., data) for each member of the cohort of new members. The contact data can include name, phone number, email, physical address, etc.

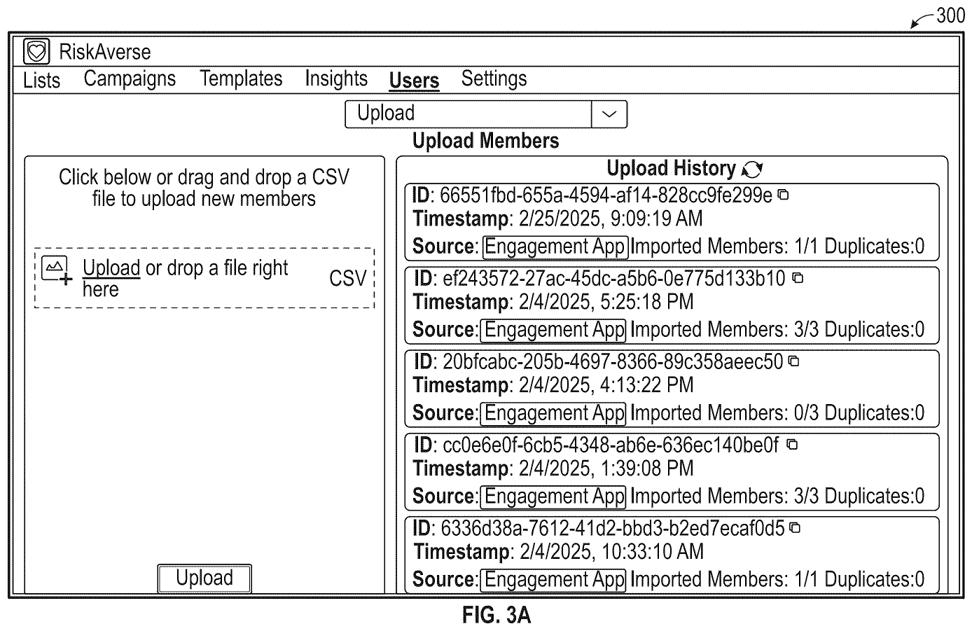

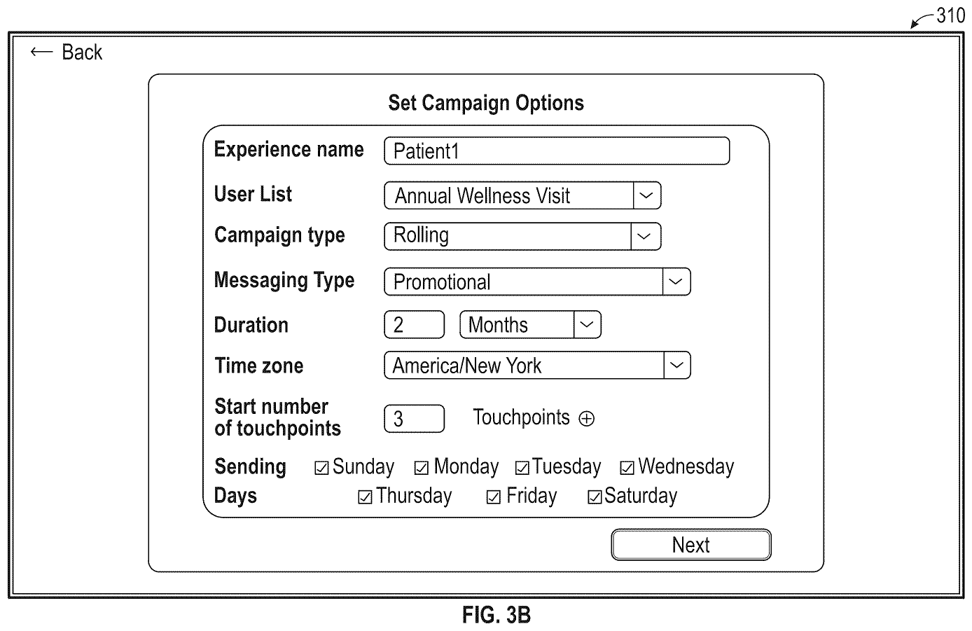

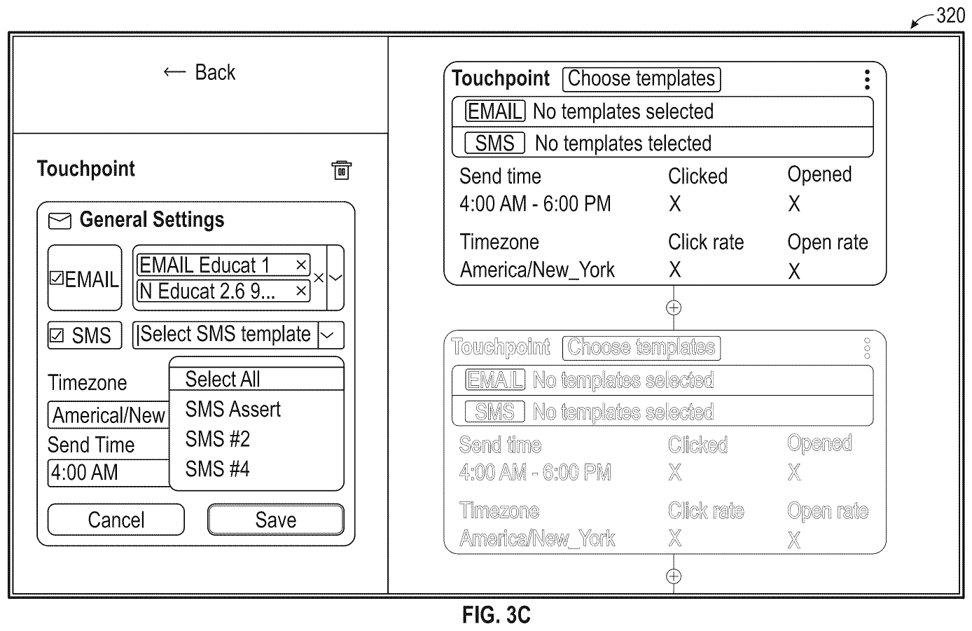

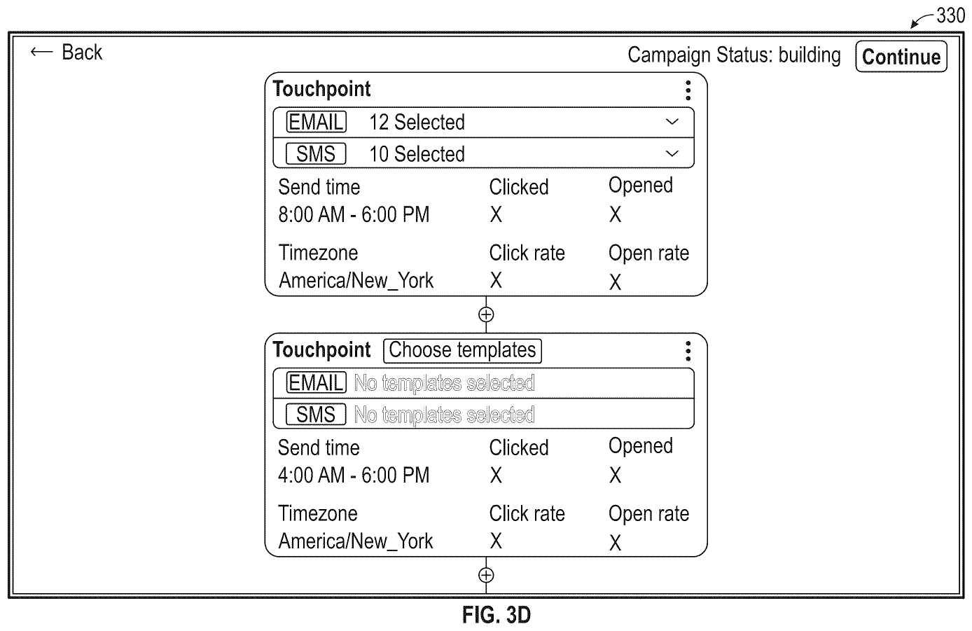

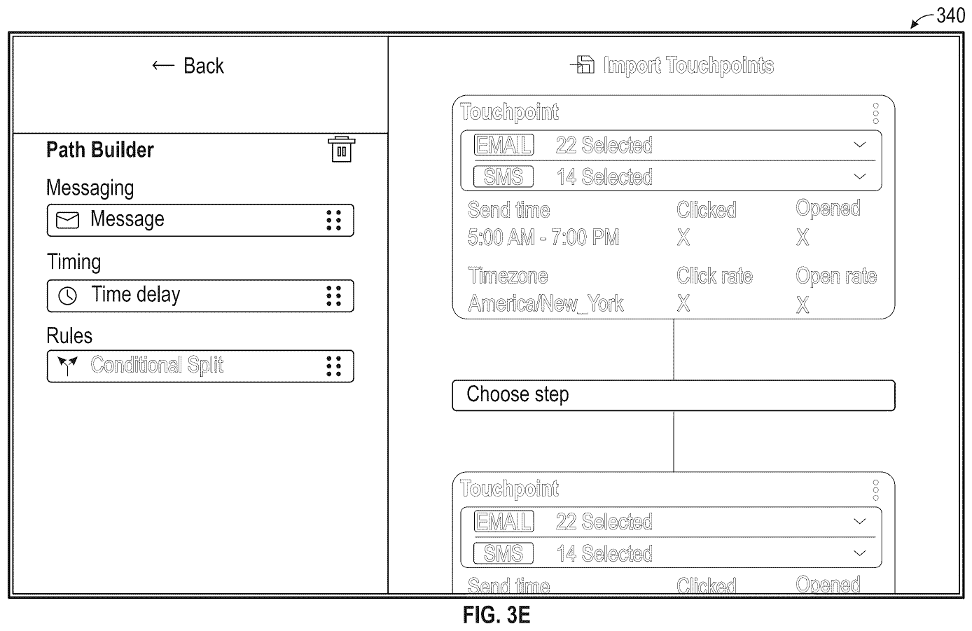

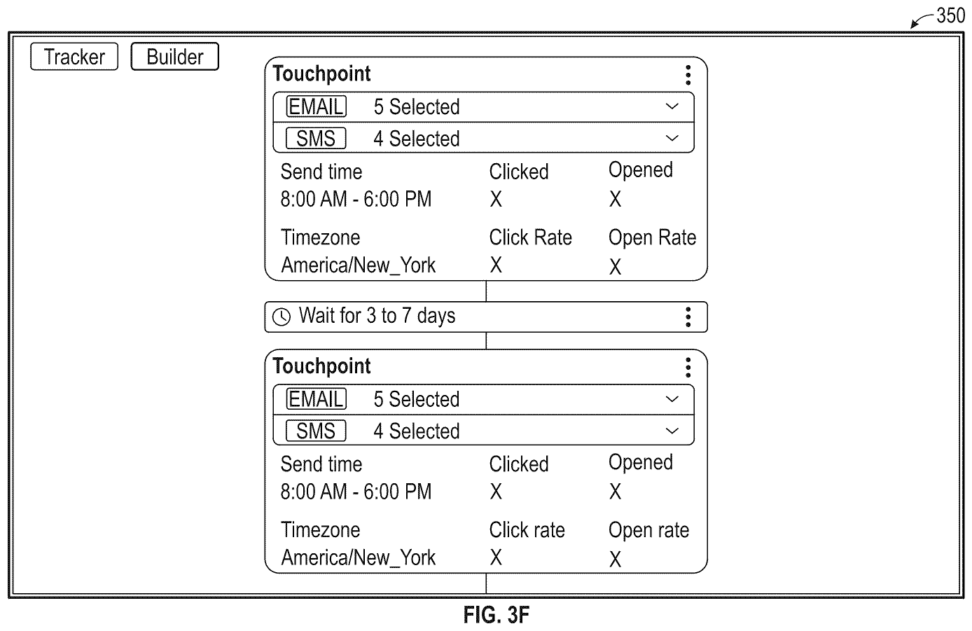

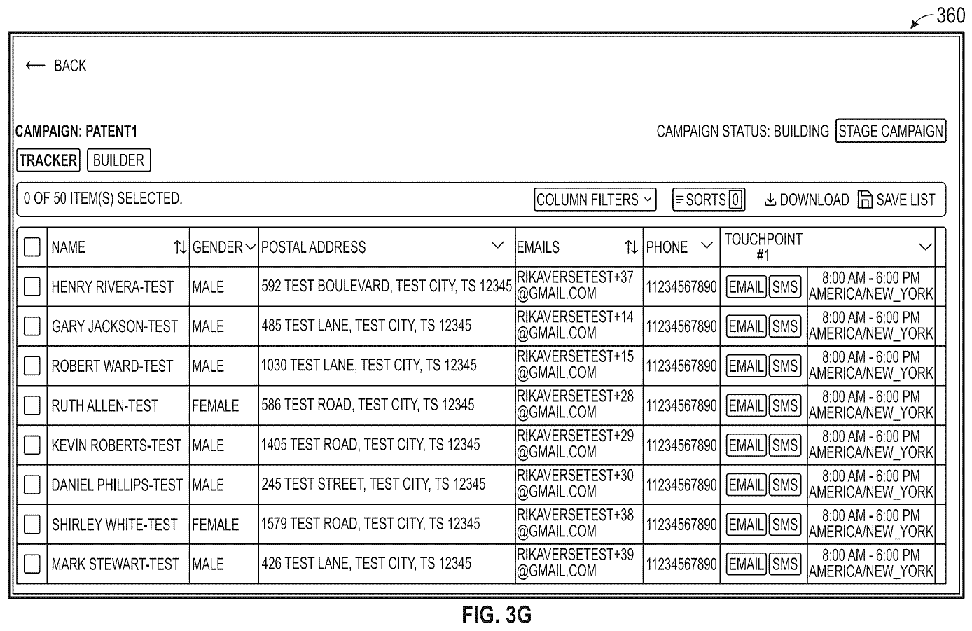

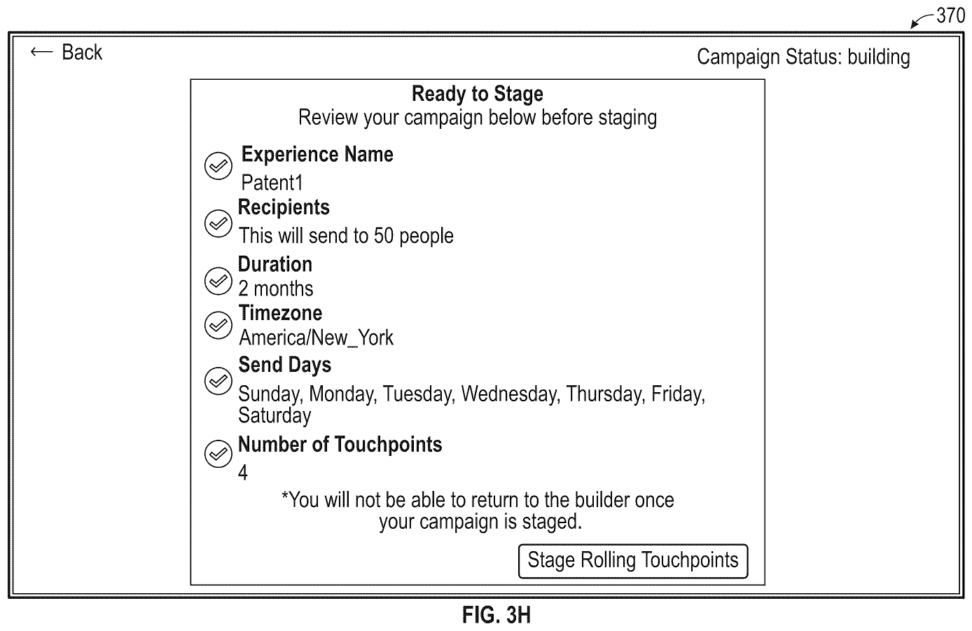

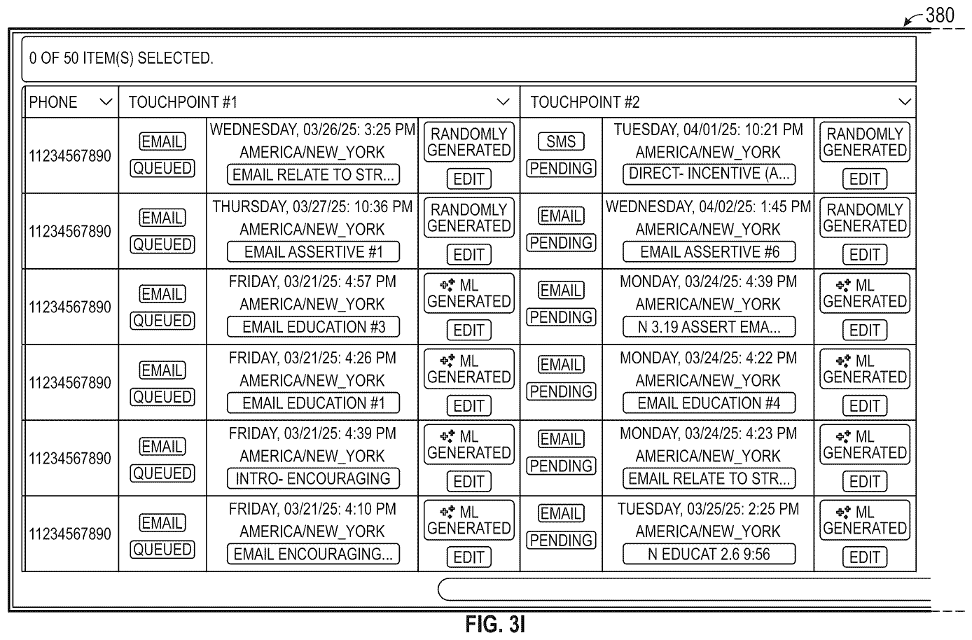

The engagement application UI 102 can be the user interface for facilitating interactions with the API 104. For example, the engagement application UI 102 can include interfaces for creating and managing lists of members, messaging templates, and/or messaging campaigns, as shown in and described with respect to the graphical user interfaces (GUIs) in FIGS. 3A-3I. As described herein, messaging campaigns can utilize the API 104 (and the AI model 106) to determine the preferred messaging parameters and delivery times to use to engage individual members in a list. In some examples, constraints such as time of day and day of week constraints can be provided by the user (e.g., vendor) as input at the engagement application UI 102 to modify the campaign. In some examples, the engagement application UI 102 can also provide graphical user interfaces to view information on messages that have been sent out to members, such as delivery status and following engagements (click, open, etc.).

When new member data is received by the engagement application UI 102, an entry in the member database 108 can be created for each member in the new member data. The entry can contain all relevant information (e.g., healthcare data including demographic information, etc.) about the member. If a member has already been added and is on another active campaign list, they can be automatically included in the relevant member lists. For example, if a member is enrolled for a screening service and, three months later, is identified for an annual wellness visit, they can be seamlessly added to the appropriate list and campaign.

The engagement application UI 102 can pass the new healthcare data to the API 104. The API 104 can facilitate interactions between the engagement application UI 102 and the AI model 106 itself. For example, the API 104 can receive requests from the engagement application UI 102 and can return optimal outreach parameters based on the output from the AI model 106 for each member indicated in the request, as described in greater detail below.

The API 104 can pass the new healthcare data to the AI model 106. The AI model 106 can include one or more AI models. In some examples, the AI model 106 can include a logistic regression model, neural network, random forest classification model, etc. As described in greater detail with respect to FIG. 2, the AI model 106 can be trained based on historical healthcare data and engagement result data of a population of historical members. In particular, in some examples, the AI model 106 can be trained based at least on the historical demographic data of the population of historical members. The historical healthcare data can be stored in and retrieved from the member database 108.

The member database 108 can store demographic data, healthcare (e.g., medical) data, and/or any additional metadata for each member. In some examples, within the member database 108, each member can have a unique identifier. In some examples, each member can be tied to a specific client organization. In some examples, different client organizations can be split amongst different databases or can be contained within the same database. The amount of information contained within the member database 108 for each member may vary based on the amount of information received from the member (e.g., via the vendor). For example, certain fields such as the member's phone number, email address, and/or address may not be present based on the vendor and/or member's preferences. The AI model 106 may not require said fields of data for determining the optimal outreach parameters for the member.

The historical healthcare data can include clinical data, demographic data, geographic data, insurance claim data, behavioral data, psychographic data, and/or social determinants of health (SDoH) data of a population of historical members. As described herein, demographic data can include age, gender, race or ethnicity, marital status, and/or education level of the population of historical members. Examples of each of these types of data are described in greater detail below with respect to FIG. 2.

The engagement result data for training the AI model 106 can be stored in the engagement database 110. The engagement result data can indicate whether the plurality of historical members engaged with the outreach message, and the parameters by which the message was generated and deployed. For example, the engagement result data can indicate a time at which the plurality of historical members engaged with the outreach message and a time at which the outreach message was sent.

The engagement database 110 can store information about historical individual email and text message sends. In some examples, the engagement database 110 stores member lists, messaging templates and/or messaging campaigns. Data stored about each historical message (e.g., email or text) can include timestamps, such as when the message was scheduled and sent, information about the contents of the message itself, etc. In some examples, the associated template for the message that was sent can be stored in the engagement database 110. In some examples, details and metadata about member lists and messaging templates can be stored in the engagement database 110. In some examples, recipient and/or sender addresses/phone numbers can be stored in the engagement database 110. In some examples, the campaign associated with the message send can be stored. The campaign can be broken down into a sequence of “touchpoints,” each of which can represent a single message sent to some or all of the members who are recipients in the campaign. Thus, each touchpoint may have message data associated with it that can be stored in the engagement database 110.

The AI model 106 can determine a combination of optimal output parameters for targeting and engaging the new member. As described in greater detail below with respect to FIG. 2, the AI model 106 can determine the combination of optimal outreach parameters for targeting and engaging the new member based on the new healthcare data of the new member, and the historical healthcare data and engagement result data of the population of historical members. The AI model 106 itself can provide a binary output, i.e., a “success” or a “failure” given a set of input parameters. The AI model 106 can generate a confidence score for that prediction to allow the API 104 to prioritize certain outreach combinations. The optimal set of input parameters can be generated by the API 104 or the AI model 106 based on the outreach combinations and assigned confidence scores. The combination of optimal output parameters can include a tone of the outreach message, a time at which to send the outreach message, a channel through which to send the outreach message, and/or a cadence at which to send the outreach message.

Once the AI model 106 determines the combination (or several combinations) of optimal output parameters, the AI model 106 can pass the combination(s) to the API 104. In some examples, the API 104 can sanitize the output from the AI model 106 based on the given constraints of the request (e.g., time constraint, message channel constraint etc.). In examples in which the AI model 106 is a binary classifier, the API 104 can test relevant combinations of outreach parameters to determine a set of outreach parameters that will produce a positive result with the highest degree of confidence. Testing the relevant combinations can include calculating all the possibilities of message parameters, and then ranking the sets of parameters that result in “success” predictions by confidence score. Once the combination of optimal outreach parameters has been determined, the API 104 can respond to the request received from the engagement application UI 102 with the combination of optimal outreach parameters for engaging the new member.

The API 104 can pass the combination of optimal output parameters to the engagement application UI 102. The engagement application UI 102 can generate an outreach message for engaging the new member. The engagement application UI 102 can generate the outreach message based on at least one message constraint received from a user of the UI 102 and the combination of optimal outreach parameters. The message constraint can include the content of the message. Additionally, or alternatively, the message constraint can include a constraint on a time at which to send the outreach message, a constraint on a channel through which to send the outreach message, a constraint on a tone of the outreach message, and/or a constraint on a cadence at which to send the outreach message. In some examples, based on the combination of optimal output parameters, the engagement application UI 102 can determine a template to use for the content of the message. The content of the message can be chosen from a list of predefined templates managed by the engagement application UI 102. In some examples, the templates can also be tagged with a tone based on the message content. Thus, the template can store message content and/or appropriate tone category.

The engagement application UI 102 can deploy the outreach message to the new member. The engagement application UI 102 can deploy the outreach message based on the combination of optimal outreach parameters that targets the new member regarding healthcare information. Healthcare information can include information regarding benefits, such as a medical benefit, a health insurance benefit, a vision benefit, a hearing benefit, a financial benefit, an educational benefit, and/or a life insurance benefit. The outreach message can be an email, text (e.g., SMS), or postal mail message. In some examples, the engagement application UI 102 can leverage a third-party service and/or an internal service to send the message to the member. For example, the engagement application UI 102 can use an API call with the third-party service and/or internal service to send the message to the member.

Once the message has been deployed, the system 100 can receive an engagement result from the new member. The system 100 can store the engagement result in the engagement result database 110. The engagement result of the new member may be stored such that it is associated with the optimal parameters for engaging the new member, and the healthcare data of the new member. In this way, the AI model 106 can be continuously retrained based on the engagement results of members.

In some examples, based on a determination that the new member engaged with the outreach message, the system 100 can deploy at least one additional outreach message to the new member based on the combination of optimal outreach parameters. For example, the initial deployed message may be part of a campaign of messages intended to be sent to the new member, and the parameters of the subsequent messages may be sent in accordance with the optimal parameters (and the at least one message constraint) of the initial message.

In some examples, based on a determination that the new member did not engage with the outreach message, the system 100 can determine a new combination of optimal outreach parameters for engaging the new member using the AI model 106. The new combination of optimal outreach parameters can be based on the new healthcare data of the new member, the historical healthcare data, and the engagement result data. The new combination of optimal outreach parameters can be different from the initial combination of optimal outreach parameters.

In accordance with the new combination of optimal outreach parameters, the system 100 (i.e., the engagement application UI 102) can generate a new outreach message for engaging the member. The new outreach message can further be based on the at least one message constraint previously received at the system 100. Based on the new combination of optimal outreach parameters, the engagement application UI 102 can deploy the new outreach message to the new member that targets the new member regarding the healthcare information. The above process regarding deploying additional messages in accordance with the optimal outreach parameters if the new member engages with the outreach message, or determining new parameters for engaging the new member, can be continuously repeated for the duration of a campaign.

The system 100 can generate different optimal parameters and related outreach messages for each new member. For example, the engagement application UI can receive new healthcare data for a first member (“Member A”) and a second member (“Member B”). For Member A, the AI model 106 can generate a first combination of optimal outreach parameters based on the new healthcare data of Member A, the historical healthcare data, and the engagement result data. For Member B, the AI model 106 can generate a second combination of optimal outreach parameters based on the new healthcare data of Member B, the historical healthcare data, and the engagement result data. The combination of optimal outreach parameters for Member A can be different from the combination of optimal outreach parameters for Member B. For example, the time at which to send a message, a channel through which to send the message, a tone of the message, and/or a cadence at which to send the message can be different between Member A and Member B.

The system 100 can generate first outreach message for engaging Member A based on at least one message constraint and the combination of optimal outreach parameters for Member A. The system 100 can generate a second outreach message for engaging Member B based on at least one message constraint and the combination of optimal outreach parameters for Member B. The outreach message for Member A can be different from the outreach message for Member B. For example, the tone of the message for Member A can be different from Member B.

The system 100 can deploy the outreach message for Member A to Member A based on the combination of optimal outreach parameters that targets Member A regarding healthcare information. The system 100 can deploy the outreach message for Member B to Member B based on the combination of optimal outreach parameters that targets Member B regarding healthcare information. The time, channel, cadence, etc. according to which the outreach message for Member A is deployed can be different from the time, channel, cadence, etc. according to which the outreach message for Member B is deployed.

FIG. 2 depicts a training process 200 of the AI model 106 shown in and described with respect to FIG. 1. The training process 200 can begin with data collection 202. Data collection 202 can include ingesting historical healthcare data and engagement result data. Historical healthcare data and engagement result data are illustrated collectively as data 204 to demonstrate the association between the historical healthcare data and the engagement result data. As described herein, a datapoint for training the AI model can be based on a single message deployed to a member. The datapoint can include healthcare data of the member and engagement result data.

Engagement result data can include an indication of a member engaged with the deployed message, and the parameters the message was generated and deployed in accordance with. The indication can be a categorical indication such as “success” or “failure.” In some examples, the indication is a Boolean value (e.g., 1 or 0), or the categorical indication is associated with a Boolean value.

Historical healthcare data can include clinical data, demographic data, geographic data, insurance claim data, behavioral data, psychographic data, and/or social determinants of health (SDoH) data of a population of historical members. In some examples, at minimum, the historical healthcare data includes demographic data.

Demographic data can include but are not limited to age, gender, race or ethnicity, marital status, and/or education level. Geographic data can include but are not limited to residential ZIP code, neighborhood classification (e.g., urban, suburban, rural), distance from the nearest healthcare facility, census tract, county of residence, air pollution rates, and/or neighborhood walkability scores. Clinical data can include but are not limited to diagnosed chronic condition (e.g., diabetes, hypertension, screenings, etc.), medication history, laboratory test results, imaging reports (e.g., X-rays, MRIs), and/or immunization records. Insurance claim data can include but are not limited to insurance plan type, billing codes (e.g., CPT, ICD-10, etc.), date of service, payment amount, and/or denied claim reason. Behavioral data can include but are not limited to smoking status, alcohol consumption frequency, physical activity level, adherence to prescribed medication, and/or dietary habits. Psychographic data can include but is not limited to health motivation level (e.g., proactive, reactive), preferred communication style (e.g., email, phone), attitudes toward preventive healthcare, perception of healthcare system trustworthiness, and/or wellness goals (e.g., weight loss, mental health). Social determinants of health data can include but are not limited to housing stability (e.g., temporary housing, homeownership), employment status, access to transportation, availability of healthy food options in the area, and/or neighborhood safety concerns.

Data collection 202 can include collecting new healthcare data 206 for one or more new members. The new healthcare data 206 can include categorically similar data as the historical healthcare data 204, but for a cohort of new members. For example, the new healthcare data can include clinical data, demographic data, geographic data, insurance claim data, behavioral data, psychographic data, and/or social determinants of health (SDoH) data of a population of historical members. In some examples, at minimum, the new healthcare data 206 includes demographic data. In some examples, the new healthcare data 206 includes clinical data and/or demographic data.

In some examples, the data ingested by the AI model is split to normalize the datasets. A portion of the data can be used for training the AI model, and the other portion of the data can be used to test or validate the models. For example, the data can be split 80:20, i.e., 80% of the data may be used for training, and the other 20% may be used for testing/validation. Other splits of data are also possible, e.g. 70:30, 75:25, 85:15, 90:10, or 95:5, for example.

In some examples, the datasets can be weighted and/or artificial datasets can be created to balance the number of data points marked as “success” or “failure.” When dealing with real world data, on average, people are more likely to ignore a message than interact with it. Therefore, in the training datasets of the AI model, there may be more failures (messages that did not get interacted with) than successes. For experimentation purposes, it can be beneficial to train a model on a dataset that is more balanced. Thus, some iterations of the AI model can be trained on datasets that have been artificially balanced. Artificial balancing can include the creation of “successful” datasets based on extrapolations of other datasets, until the number of successes and failures is equal. In some examples, some datasets can be given a higher “weight” to achieve a similar effect.

The data collected at data collection 202 can be used to determine the combination of optimal outreach parameters for each new member at model training 208. Model training may involve the AI model(s) (e.g., AI model 106) and the machine learning API (e.g., API 104) that sanitizes the output from the AI model. The AI model can be trained on historical healthcare data, engagement result data, and new member healthcare data.

In some examples, determining the combination of optimal outreach parameters includes using a random forest classification model to determine a plurality of combinations of outreach parameters for each member. For each combination of parameters, the classification model can generate one output (e.g., yes or no, success or failure, etc.). As a random forest model, the classification model can include many decision trees that each generate one output (yes/no, success/failure, etc.). Each tree can be trained on a random subset of the parameters; thus, each tree may not take every parameter into account. A machine learning library can automatically select different subsets of parameters for each decision tree, making the AI model more robust and less biased. The output of the classification model (i.e., the plurality of combinations for each member) can be based on which outcome (e.g., yes/no) most decision trees chose. From the output of the classification model, a plurality of confidence scores can be assigned to the plurality of combinations of outreach parameters. The API can assign the confidence scores. The confidence score can otherwise be understood to be the percentage of decision trees that voted for the combination of outreach parameters. For example, a low confidence score (e.g., 0.55) means only 55% of trees agreed on it, whereas a high score (e.g., 0.97) means 97% of decision trees agreed on it. The plurality of confidence scores can be compared to determine the optimal combination of outreach parameters. In some examples, the API can compare the confidence scores to determine the optimal combination of outreach parameters.

Model training 208 can include an AI model based on gradient boosting (e.g., Extreme Gradient Boosting (XGBoost) or similar). The AI model may be a binary classification model. Other AI model may be used, such as logistic regression, neural networks, random forest classification, etc. Training the AI model may include varying the hyperparameters of the AI model. For example, the depth, child weight, and estimators (i.e., number of trees) of the AI model. Controlling the depth, regularization, and number of trees of the AI model can achieve an optimal balance between accuracy and generalization.

Model training 208 can include analyzing the output of the AI model and tuning the hyperparameters. For example, parameters such as learning rate, tree depth, regularization terms, and/or boosting rounds of the AI model can be optimized to enhance the AI model's predictive power. In this way, the accuracy, area under the curve (AUC), and/or the F1 score of the AI model can be improved. The accuracy of the model can be used for balanced datasets to measure the proportion of correctly classified instances among all predictions. The AUC can be used in imbalanced datasets to measure the ability of the model to distinguish between classes. A higher AUC can indicate better discrimination. The F1 score can be used for imbalanced datasets to measure the harmonic mean of precision and recall of the model. Model training 208 can be repeated one or more times with the adjusted parameters to improve the performance of the AI model.

The outreach parameters provided as output from the API and AI model can include at least one of a tone of the outreach message, a time at which to send the outreach message, a channel through which to send the outreach message, and/or a cadence at which to send the outreach message. In a non-limiting example, a tone of the outreach message can be assertive, a time at which to send the outreach message can be 8:02 AM Eastern Time Zone, a channel through which to send the outreach message can be SMS, and/or a cadence at which to send the outreach message can be 3 days before the next outreach message.

Any message constraints 210 from the user can be applied to the outreach parameters from the AI model to achieve the optimal output parameters for engaging the member. As described herein, message constraints 210 can include a constraint on a time at which to send the outreach message, a constraint on a channel through which to send the outreach message, a constraint on a tone of the outreach message, and/or a constraint on a cadence at which to send the outreach message. An example of time constraint may be that messages are only sent between 8 AM and 5 PM. An example of a constraint on tone may be that messages are only sent in an encouraging tone. An example of constraint on channel may be that messages are only sent via text. An example of a constraint on cadence may be that messages are only sent twice before ending the campaign.

The optimal outreach parameters provided as model output 212 can be the outreach parameters from the AI model and API when no message constraints are applied to the outreach parameters. Alternatively, the optimal outreach parameters provided as model output 212 can be the outreach parameters from the AI model and API with the message constraints applied. The model output 212 from a regression AI model can be an exact time, channel, tone, and/or cadence at which to deploy a message to the member. In another example, the model output 212 from a classification model can be a binary classification from a small finite group (yes/no) for each combination of optimal outreach parameters.

FIGS. 3A-3I show graphical user interfaces (GUIs) of a platform for interacting with the AI-backed system of FIG. 1. FIG. 3A illustrates a GUI 300 for uploading member lists into the system. In some examples, vendors can add lists either through a file upload or an API integration. The GUI 300 can also show upload history, allowing vendors to track previously added lists.

FIG. 3B shows a GUI 310 for selecting and providing the setup parameters available for configuring a member list campaign. Once a list is successfully uploaded, users can define key parameters to initiate the campaign setup process. The setup parameters can include selecting a member list, a campaign type, a messaging type, a duration, a time zone, a number of touchpoints, and/or days on which to send the message.

FIG. 3C shows GUI 320 for selecting additional setup options for individual touchpoints within a campaign. These options can include toggling communication channels on or off, selecting specific messages for each channel, configuring time zones, and/or setting the AI-driven delivery window to optimize engagement.

FIG. 3D shows GUI 330 of the campaign setup process, highlighting the system's warning messages. The warning messages can notify users of any missing or incomplete configurations. In this example, a warning is triggered due to missing parameters (shown in GUI 320 of FIG. 3C) for the second touchpoint.

FIG. 3E shows GUI 340 of an overview of the campaign journey, summarizing all configured steps. The GUI 340 can allow users to edit the journey after its initial creation.

FIG. 3F shows GUI 350 for setting advanced customization options, including the ability to add forced time delays between touchpoints. As shown, users can configure conditional splits based on various user actions. For example, a conditional split may trigger based on an SMS click, an email click, an email open, and/or external feedback from a partner system confirming successful program enrollment.

FIG. 3G shows a GUI 360 of a summary of all individuals on the target list, along with their associated touchpoint parameters, before the campaign is launched.

FIG. 3H shows a GUI 370 of the campaign launch process, including an overview of the campaign parameters. From GUI 370, a vendor can activate the configured campaign.

FIG. 3I shows a GUI 380 of an overview of all deployed campaigns, categorized by member lists. The systems can track the status of each touchpoint deployment. For example, this can include but is not limited to a touchpoint being in the pending status, the delivered status, the queued status, etc. The GUI 380 can be used to track deliverability and ensure the systems are operating properly. The GUI 380 can provide visibility into active and past engagements.

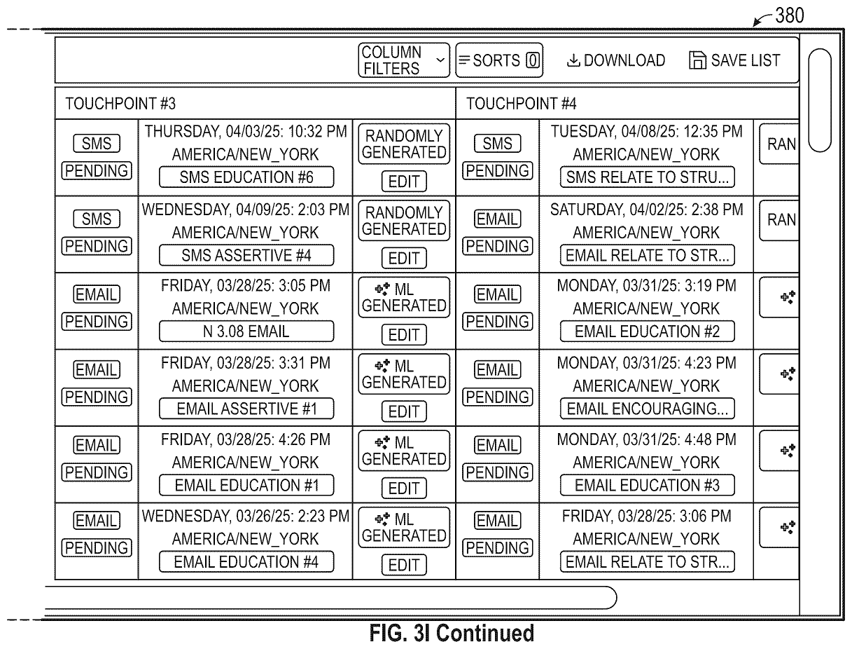

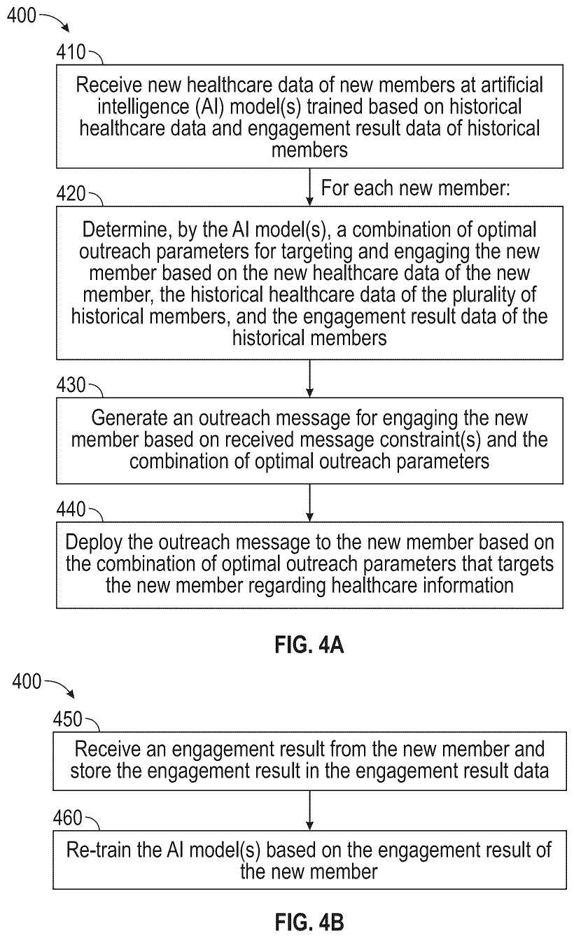

FIG. 4A shows an automated method 400 for targeted outreach messaging new members regarding healthcare information. At block 410, the method 400 can include receiving new healthcare data of one or more new members at one or more artificial intelligence (AI) models trained based on historical healthcare data and engagement result data of a plurality of historical members. In some examples, the AI model is further trained based at least on historical demographic data of the plurality of historical members.

The method 400 can include, for each outreach message to be sent to a particular member, a series of steps (indicated by blocks 420-440 in FIG. 4A). At block 420, the method 400 can include determining, by the one or more AI models, a combination of optimal outreach parameters for targeting and engaging the new member based on the new healthcare data of the new member, the historical healthcare data of the plurality of historical members, and the engagement result data of the plurality of historical members. In some examples, determining the combination of optimal outreach parameters for targeting and engaging the new member includes using a random forest classification AI model configured for determining a plurality of combinations of outreach parameters for engaging the new member. In some examples, determining the plurality of optimal outreach parameters for targeting and engaging the new member includes assigning a plurality of confidence scores to the plurality of combinations of outreach parameters, and comparing the plurality of confidence scores to determine the combination of optimal outreach parameters.

At block 430, the method 400 can include generating an outreach message for engaging the new member based on at least one received message constraint and the combination of optimal outreach parameters.

At block 440, the method 400 can include deploying the outreach message to the new member based on the combination of optimal outreach parameters that targets the new member regarding healthcare information.

In some examples, method 400 includes retraining the AI model based on the new member's engagement with the deployed message, shown in FIG. 4B. At block 450, the method 400 can include receiving an engagement result from the new member and storing the engagement result in the engagement result data. At block 460, the method 400 can include re-training the AI model based on the engagement result of the new member.

In some examples, the method 400 can include, based on a determination that the new member engaged with the outreach message, deploying at least one additional outreach message to the new member based on the combination of optimal outreach parameters.

In some examples, the method 400 can include, based on a determination that the new member did not engage with the outreach message, determining, by the AI model(s), a new combination of optimal outreach parameters for targeting and engaging the new member based on the new healthcare data of the new member, the historical healthcare data, and the engagement result data. The new combination of optimal outreach parameters may be different from the initial combination of optimal outreach parameters. Accordingly, in this example, the method 400 can further include generating a new outreach message for engaging the new member based on the at least one received message constraint and the new combination of optimal outreach parameters. Additionally, the method 400 can include deploying the new outreach message to the new member based on the new combination of optimal outreach parameters that targets the new member regarding the healthcare information.

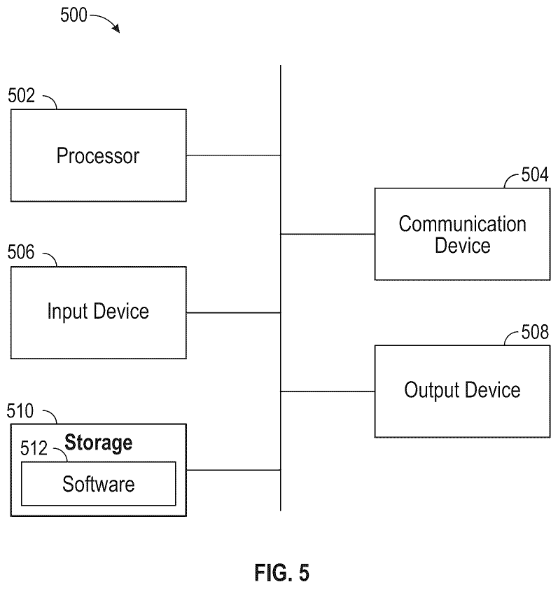

FIG. 5 depicts a computing device 500, according to one or more examples of the disclosure. In one or more examples, computing device 500 may be configured to execute the method 400 for targeted outreach messaging new members regarding healthcare information. In one or more examples, computing device 500 may be configured to generate and display graphical user interfaces (GUIs) for using an AI system for engaging members, such as the GUIs illustrated in FIGS. 3A-3I.

Computing device 500 can be a host computer connected to a network. Computing device 500 can be a client computer or a server. As shown in FIG. 5, computing device 500 can be any suitable type of microprocessor-based device, such as a personal computer, workstation, server, or handheld computing device (portable electronic device) such as a phone or tablet. The computing device 500 can include, for example, one or more of processors 502, input device 506, output device 508, storage 510, and communication device 504.

Input device 506 can be any suitable device that provides an input, such as a touch screen, keyboard or keypad, mouse, or voice-recognition device. Output device 508 can be any suitable device that provides output, such as a display, touch screen, haptics device, or speaker.

Storage 510 can be any suitable device that provides storage, such as an electrical, magnetic, or optical memory, including a RAM, cache, hard drive, or removable storage disk. Communication device 504 can include any suitable device capable of transmitting and receiving signals over a network, such as a network interface chip or device. The components of the computer can be connected in any suitable manner, such as via a physical bus or wirelessly.

Software 512, which can be stored in storage 510 and executed by processor 502, can include, for example, the programming that embodies the functionality of the present disclosure (e.g., as embodied in the devices as described above).

Software 512 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 510, that can contain or store programming for use by or in connection with an instruction execution system, apparatus, or device.

Software 512 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Computing device 500 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Computing device 500 can implement any operating system suitable for operating on the network. Software 512 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

### EXAMPLES

The following examples are merely illustrative and are not intended to limit the scope of the disclosure provided herein.

In an exemplary embodiment illustrating the AI methods and systems described herein personalizing engagement, consider three sample members from a list of 100,000 members. Member A is a 64-year-old female, low socioeconomic status (SES), has Type 2 diabetes, and has transportation access. From these characteristics, the AI system may determine the optimal engagement strategy for engaging Member A starts with an SMS at 8:02 AM on a Monday using an assertive tone (Content Template #2), followed by a second touchpoint via email at 2:27 PM on Friday, four days later, with a tone that relates to her struggles (Content Template #6). Within the bounds set by the vendor, this sequence would continue for as many touchpoints are necessary until Member A engages with the program.

Member B is a 55-year-old male, high SES, overweight but without Type 2 diabetes, and with transportation access. Using this information, the AI system determines the optimal engagement strategy for engaging Member B may start with a first touchpoint at 6:45 PM on Wednesday via email, using an informative and professional tone (Content Template #4). His second touchpoint can occur at 10:15 AM the following Tuesday through SMS, using a motivational tone (Content Template #1).

Member C is a 37-year-old female, medium SES, no chronic conditions, but lacking transportation. The AI system can determine the optimal engagement strategy for engaging Member C involves the first touchpoint at 12:05 PM on a Saturday via SMS with a friendly and empathetic tone (Content Template #3), and her second touchpoint can be a phone call at 9:00 AM the next Thursday, using a supportive tone that emphasizes accessibility options (Content Template #5).

Each member's outreach can be dynamically tailored based on the learnings of the AI system from past information ingested by the AI systems. For example, the AI systems can identify that the combination of Type 2 diabetes and low SES may lend itself to outreaching through a specific channel or time. Uploading a list of members (e.g., a list of 100,000 members including Members A, B, and C), determining the optimal parameters for engaging each of the members, and deploying a campaign in accordance with those parameters takes place within minutes, at the click of a button.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

For the purpose of clarity and a concise description, features are described herein as part of the same or separate examples; however, it will be appreciated that the scope of the disclosure includes examples having combinations of all or some of the features described.

What is claimed is:

1. An automated method for targeted outreach messaging new members regarding healthcare information, comprising:
receiving new healthcare data of one or more new members at one or more artificial intelligence (AI) models trained based on historical healthcare data and engagement result data of a plurality of historical members; and
for each outreach message:
determining, by at least the one or more AI models, a combination of optimal outreach parameters for targeting and engaging the new member based on the new healthcare data of the new member, the historical healthcare data of the plurality of historical members, and the engagement result data of the plurality of historical members;
generating an outreach message for engaging the new member based on at least one received message constraint and the combination of optimal outreach parameters;
and deploying the outreach message to the new member based on the combination of optimal outreach parameters that targets the new member regarding healthcare information.

2. The method of claim 1, wherein the new healthcare data and/or the historical healthcare data comprises clinical data including at least one medical condition, a medication history, at least one laboratory test result, at least one imaging report, and/or an immunization record.

3. The method of claim 1, wherein the combination of optimal outreach parameters comprises a tone of the outreach message, a time at which to send the outreach message, a channel through which to send the outreach message, and/or a cadence at which to send the outreach message.

4. The method of claim 1, wherein the one or more AI models are further trained based at least on historical demographic data of the plurality of historical members.

5. The method of claim 1, wherein the one or more new members are different from the plurality of historical members.

6. The method of claim 1, wherein the engagement result data indicates a time at which the plurality of historical members engaged with the outreach message and a time at which the outreach message was sent.

7. The method of claim 1, wherein the at least one received message constraint comprises a content of the outreach message.

8. The method of claim 7, wherein the at least one received message constraint comprises a constraint on a time at which to send the outreach message, a constraint on a channel through which to send the outreach message, a constraint on a tone of the outreach message, and/or a constraint on a cadence at which to send the outreach message.

9. The method of claim 1, wherein determining the combination of optimal outreach parameters for targeting and engaging the new member comprises using a random forest classification AI model configured for determining a plurality of combinations of outreach parameters for engaging the new member.

10. The method of claim 9, wherein determining the combination of optimal outreach parameters for targeting and engaging the new member comprises, by a machine learning application programming interface (API) and/or the one or more AI models:
assigning a plurality of confidence scores to the plurality of combinations of outreach parameters; and
comparing the plurality of confidence scores to determine the combination of optimal outreach parameters.

11. The method of claim 1, comprising:
receiving an engagement result from the new member; and
storing the engagement result in the engagement result data.

12. The method of claim 11, comprising re-training the one or more AI models based on the engagement result of the new member.

13. The method of claim 11, comprising, based on a determination that the new member engaged with the outreach message, deploying at least one additional outreach message to the new member based on the combination of optimal outreach parameters.

14. The method of claim 11, comprising, based on a determination that the new member did not engage with the outreach message:
determining, by the one or more AI models, a new combination of optimal outreach parameters for targeting and engaging the new member based on the new healthcare data of the new member, the historical healthcare data, and the engagement result data, wherein the new combination of optimal outreach parameters is different from the initial combination of optimal outreach parameters;
generating a new outreach message for engaging the new member based on the at least one received message constraint and the new combination of optimal outreach parameters; and
deploying the new outreach message to the new member based on the new combination of optimal outreach parameters that targets the new member regarding the healthcare information.

15. The method of claim 1, comprising:
receiving new healthcare data of a first member and a second member of the one or more new members at the one or more AI models;
determining, by the one or more AI models, a first combination of optimal outreach parameters for targeting and engaging the first member based on the new healthcare data of the first member, the historical healthcare data, and the engagement result data;
generating a first outreach message for engaging the first member based on the at least one received message constraint and the first combination of optimal outreach parameters;
deploying the first outreach message to the first member based on the combination of optimal outreach parameters that targets the first member regarding healthcare information;
determining, by the one or more AI models, a second combination of optimal outreach parameters for targeting and engaging the second member based on the new healthcare data of the second member, the historical healthcare data, and the engagement result data, the second combination of optimal outreach parameters different from the first combination of optimal outreach parameters;
generating a second outreach message for engaging the second member based on the at least one received message constraint and the second combination of optimal outreach parameters, the second outreach message different from the first outreach message; and
deploying the second outreach message to the second member based on the combination of optimal outreach parameters that targets the second member regarding healthcare information.

16. The method of claim 1, wherein the healthcare information comprises information regarding at least one of a medical benefit, a health insurance benefit, a vision benefit, a hearing benefit, a financial benefit, an educational benefit, and a life insurance benefit.

17. The method of claim 1, wherein the new healthcare data and/or the historical healthcare data comprises at least one of demographic data, geographic data, insurance claim data, behavioral data, psychographic data, and social determinants of health (SDoH) data.

18. The method of claim 1, wherein receiving the new healthcare data for the one or more new members at the one or more AI models comprises:
receiving the new healthcare data at an engagement application user interface (UI);
passing the new healthcare data from the engagement application UI to a machine learning application programming interface (API); and
passing the new healthcare data from the machine learning API to the one or more AI models.

19. The method of claim 18, comprising, following passing the new healthcare data from the machine learning API to the one or more AI models:
passing a plurality of combinations of optimal output parameters from the one or more AI models to the machine learning API; and
passing the combination of optimal output parameters determined from the plurality of combinations of optimal output parameters from the machine learning API to the engagement application UI that generates the outreach message for engaging the new member and deploys the outreach message to the new member.

20. A system for targeted outreach messaging new members regarding healthcare information, comprising one or more processors; memory; and one or more programs stored on the memory that when executed by the one or more processors cause the one or more processors to:
receive new healthcare data of one or more new members at one or more artificial intelligence (AI) models trained based on historical healthcare data and engagement result data of a plurality of historical members; and
for each outreach message:
determine, by at least the one or more AI models, a combination of optimal outreach parameters for targeting and engaging the new member based on the new healthcare data of the new member, the historical healthcare data of the plurality of historical members, and the engagement result data of the plurality of historical members;
generate an outreach message for engaging the new member based on at least one received message constraint and the combination of optimal outreach parameters; and
deploy the outreach message to the new member based on the combination of optimal outreach parameters that targets the new member regarding healthcare information.

21. A non-transitory computer-readable storage medium storing one or more programs for targeted outreach messaging new members regarding healthcare information, the programs for execution by one or more processors of an electronic device that when executed by the device, cause the device to:
receive new healthcare data of one or more new members at one or more artificial intelligence (AI) models trained based on historical healthcare data and engagement result data of a plurality of historical members; and
for each outreach message:
determine, by at least the one or more AI models, a combination of optimal outreach parameters for targeting and engaging the new member based on the new healthcare data of the new member, the historical healthcare data of the plurality of historical members, and the engagement result data of the plurality of historical members;
generate an outreach message for engaging the new member based on at least one received message constraint and the combination of optimal outreach parameters; and
deploy the outreach message to the new member based on the combination of optimal outreach parameters that targets the new member regarding healthcare information.
